(19) United States
(12) Patent Application Publication
(10) Pub. No.: US 2026/0000895 A1
(43) Pub. Date: Jan. 1, 2026

(54) NEUROMODULATION SYSTEM TO INDUCE CHANGES IN GLYCEMIC REGULATORY SYSTEM PLASTICITY

(71) Applicant: Reshape Lifesiences, Inc., Irvine, CA (US)

(72) Inventors: Jonathan J. Waataja, Chanhassen, MN (US); Raj Nihalani, Irvine, CA (US)

(21) Appl. No.: 19/144,816

(22) PCT Filed: Dec. 29, 2023
(86) PCT No.: PCT/US2023/086459

§ 371 Date: Jun. 30, 2025

Related U.S. Application Data
(60) Provisional application No. 63/477,996, filed on Dec. 30, 2022.

(51) Int. Cl. A61N 1/36; A61N 1/372

(52) U.S. Cl. CPC A61N 1/36139; A61N 1/36053; A61N 1/3615; A61N 1/36171; A61N 1/36178; A61N 1/37217; A61N 1/37235

(57) ABSTRACT

Current Type 2 Diabetes Mellitus treatments include life-style management, pharmaceuticals and may include bariatric surgery for the morbidly obese. However, issues with compliance, side effects and permanent anatomical alterations provide a need for an alternative therapy to modulate a peripheral nerve, such as the vagus nerve, in a manner that causes a plastic change in the glycemic regulatory system, with or without the involvement of the central nervous system (CNS), that changes the activity of an organ and thereby changing the activity of that organ. Low energy vagus nerve neuromodulation has potential as a new treatment for type 2 diabetes mellitus by inducing plasticity of the body's glycemic regulatory system with low energy transient stimulation. This will allow for effective use of a small implantable pulse generator that requires infrequent, or no, charging during the life of the device.

### CROSS-REFERENCE TO RELATED APPLICATION

This application is being filed on Dec. 29, 2023, as a PCT International Patent Application and claims priority to and the benefit of U.S. Provisional Application Nos. 63/477,996, filed on Dec. 30, 2022, the disclosure of which is hereby incorporated by reference herein in its entireties.

### INTRODUCTION

It is the scope of this invention to excite a peripheral nerve in a manner that causes a plastic change in the glycemic regulatory system, including, but not limited to, the involvement of the central nervous system (CNS), that changes the activity of an organ, or organs, involved in maintaining glycemic control.

### SUMMARY

Diabetes is the 7th leading cause of US deaths with 252,000 deaths directly, or indirectly attributable to the disease. Approximately 31 million Americans suffer from Type 2 diabetes mellitus (T2DM), with 1.5 million new cases diagnosed annually. Diabetic comorbidities place a huge burden on public health care due to long-term detrimental effects on the heart, blood vessels, eyes, kidneys, and nerves. 97.5% of diabetics have at least one comorbid condition and 88.5% of diabetics have at least two comorbid condition.

The annual US T2DM economic burden is $237 B in direct treatment costs with individuals having annual expenditures of $9,600 directly attributable to the disease.

Metformin is a first-line pharmaceutical therapy. Other drugs include sulfonylureas, thiazolidinedione, DPP-4 inhibitors, SGLT2 inhibitors and GLP-1 receptor antagonists. Treatment durability is reduced 21% to 34% over 5 years⁵, requiring addition of second and third drugs. Drug therapies may induce side effects such as hypoglycemia heart failure, ketoacidosis, diarrhea, nausea, abdominal pain and orthopedic fracture. Moreover, the incidence of compliance is also a major issue, as approximately 50% of patients do not take medications as prescribed, including 33% of patients taking once-weekly GLP-1 receptor antagonists.

Insulin may be prescribed for type 2 diabetics who progress to a glycated hemoglobin (HbA1c)≥10% and/or blood glucose levels≥300 mg/dL. Insulin therapy is a tool to decrease blood glucose, however, there is a risk of hypoglycemia and a large portion of diabetics fears insulin injections. A study by Stotland et al found that 45% of diabetic subjects avoided injections because of anxiety, phobia and fear. Injection anxiety has been related to poorer treatment adherence, greater psychological distress, a greater incidence of diabetes-related hospitalization and a higher risk of retinopathy and neuropathy.

T2DM remission is seen in obese diabetic patients following metabolic surgery, but is only recommended for adults with a BMI of 30.0-34.9 kg/m2 and if medications are ineffective. However, remission rates were as high as 26% after 5 years, casting doubt on durability. Common metabolic surgery risks include bowel obstruction, GERD, gallstones, hernia, hypoglycemia, malnutrition and dumping syndrome. Nutrient deficiencies leading to hematologic, metabolic and neurologic disorders are reported.

Standalone Vagus nerve stimulation or blockade has demonstrated increased glycemic control, but only in the context of significant and sustained weight loss, making it unrealistic for many type 2 diabetics. Hepatic branch ligation has been proposed as a method to increase glycemic control, but it may cause negative changes in feeding behavior, increased hypoglycemic episodes, may affect liver regeneration, and can cause increased metastasis during liver cancer.

Optogenetic and chemogenetic vagal manipulation currently under investigation offer impressive neuronal specificity and may impact glycemic control, however, viral vector genetic modulation is currently clinically unrealistic. Electrical vagal neuronal stimulation has a proven clinical safety and efficacy profile for other indications (such as epilepsy) and may be a practical method of vagal modulation for glycemic control.

Induction of long-term potentiation or long-term depression of autonomic nerve reflex arcs for the treatment of disease may also be a potential therapy. The reflex arcs between sensory autonomic nerve afferents and autonomic nerve efferents which effect activity of visceral organs is an therapy that provides promise over the current widely used therapies.

Thus, there is a need for long-term T2DM therapies that provide a long-term therapy that can be modified in real-time to reduce the glucose levels in a patient, while also reducing the non-compliance of the therapeutic treatment.

### Neuromodulation System to Induce Changes in Glycemic Regulatory System Plasticity

The invention as described herein includes an alternative therapy to modulate a peripheral nerve, such as the vagus nerve, in a manner that causes a plastic change in the glycemic regulatory system, with or without the involvement of the central nervous system (CNS), that changes the activity of an organ and thereby changing the activity of that organ. Low energy vagus nerve neuromodulation has potential as a new treatment for type 2 diabetes mellitus by inducing plasticity of the body's glycemic regulatory system with low energy transient stimulation. This will allow for effective use of a small implantable pulse generator that requires infrequent, or no, charging during the life of the device.

### BRIEF DESCRIPTION OF THE FIGURES

FIG. 1(A) is an illustration of vagus nerve's hepatic branch effecting the reduction in pancreatic insulin release. FIG. 1(B) is an illustration of increased portal vein glucose concentration induces a decrease in afferent hepatic axon activity. FIG. 1(C) is an illustration of a block conduction through the hepatic branch of the vagus nerve. FIG. 1(D) is an illustration of a combination of vagal celiac branch stimulation.

FIG. 2 is an illustration of five possible sites of neuronal plasticity in the glycemic regulation reflex arc.

FIG. 3 is a graphical depiction of HFAC delivered at the hepatic branching point with concurrent stimulation at the celiac branching point increased glycemic control during an oral glucose tolerance test (OGTT) compared to sham.

FIG. 4 is a schematic representation of burst stimulation comprising two pulses in one burst

FIG. 5 is a schematic representation of burst stimulation with multiple pulses in one burst and two bursts.

FIG. 6 is a schematic representation of burst stimulation with multiple pulses and multiple bursts.

FIG. 7 is a schematic representation of burst stimulation with non-fixed intra-burst intervals

FIG. 8 is a schematic representation of burst stimulation with non-fixed inter-burst intervals.

FIG. 9 is an illustration of various neuromodulation sites on the vagus nerve.

FIG. 10(A) is a graphical depiction of Intravenous glucose tolerance test (IVGTT) in swine pre-and post-alloxan treatments with absolute values of plasma glucose in mg/dL. (B) is a bar graph showing Area under the curve (AUC) of the graphs in FIG. A. ** p<0.05, Mann-Whitney U test.

FIG. 11(A) is a graphical illustration of the same IVGTT as in FIG. 10A in swine pre-and post-alloxan treatments with % change in plasma glucose. FIG. (B) is a bar graph showing AUC of the graphs in FIG. A.

FIG. 12(A) is a graphical illustration of HFAC delivered at the hepatic branching point with concurrent stimulation at the celiac branching point altering glycemic control during an OGTT compared to sham. FIG.(B) is a bar graph showing AUC from the experiments in FIG. A.

FIG. 13(A) is a graphical illustration of HFAC delivered at the hepatic branching point with concurrent stimulation at the celiac branching point during an OGTT compared to sham. FIG. (B) is bar graph illustration of AUC from the experiments in FIG. 13(A).

FIG. 14 is a graphical illustration of HFAC delivered at the hepatic branching point with concurrent stimulation at the celiac branching point.

FIG. 15 is a bar graph showing the Fasting Plasma Glucose (FGP) before 1 min of HFAC delivered at the hepatic branching point with concurrent stimulation at the celiac branching and 1 day after the termination of the signals.

FIG. 16 is an illustration of time course of the experiments in FIG. 15.

FIG. 17 is a graphical illustration of (A) % change in plasma glucose, insulin, and heart rate (HR) FIG. (B) Blood pressure FIG. (C) Oxygen saturation.

### DETAILED DESCRIPTION

The body converts the carbohydrates from food into glucose, a simple sugar that serves as a vital source of energy. The hormones insulin and glucagon play an important role in glucose regulation. The pancreas contains a collection of cells called the Islet of Langerhans which releases both insulin and glucagon. When the body does not convert enough glucose, blood sugar levels remain high. The pancreas secretes insulin to help the cells absorb glucose, reducing blood sugar and providing the cells with glucose for energy. When blood glucose falls, cells in the pancreas secrete glucagon. Glucagon instructs the liver to convert stored glucose (i.e. glycogen) to glucose, making glucose more available in the bloodstream. Insulin and glucagon work in a cycle. Glucagon interacts with the liver to increase blood sugar, while insulin reduces blood sugar by helping the cells use glucose. Conditions associated with impaired glucose regulation include Type 2 diabetes, impaired glucose tolerance, impaired fasting glucose, gestational diabetes, and Type 1 diabetes. “Impaired glucose regulation” refers to alterations in one or more of glucose absorption, glucose production, insulin secretion, insulin sensitivity, GLP-1 regulation, and glucagon regulation.

Type 2 diabetes is a disease in which liver, muscle and fat cells do not use insulin properly to import glucose into the cells and provide energy to the cells. As the cells begin to starve for energy, signals are sent to the pancreas to increase insulin production. In some cases, the pancreas eventually produces less insulin exacerbating the symptoms of high blood sugar. Patients with Type 2 diabetes have a fasting blood (plasma) glucose of 126 mg/dl. or greater; oral glucose tolerance of 200 mg/dL or greater; and/or percentage of HbAIC of 6.5% or greater.

Despite the presence of treatments for type 2 diabetes, not all patients achieve glucose control or maintain glucose control. A patient that has not achieved glycemic control will typically have an HbAIC of greater than 7%. In some embodiments, patients are selected that continue to have problems with glycemic control even with drug treatment.

Patients with impaired glucose tolerance and/or impaired fasting glucose are those patients that have evidence of some minimal level of lack of glucose control. Patients can be naive to any treatment or are those that have been treated with one or more pharmaceutical treatments. “Pre-Diabetes” is a term that is used by the American Diabetes Association to refer to people who have a higher than normal blood glucose but not high enough to meet the criteria for diabetes. The lack of glycemic control can be determined by the fasting plasma glucose test (FPG) and/or the oral glucose tolerance test (OGTT). The blood glucose levels measured after these tests determine whether the patient has normal glucose metabolism, impaired glucose tolerance, impaired fasting glucose, or diabetes. If the patient's blood glucose level is abnormal within a specified range following the FPG, it is referred to as impaired fasting glucose (IFG); if the patient's glucose level is abnormal within a specified range following the OGTT, it is referred to as impaired glucose tolerance (IGT). A patient is identified as having impaired fasting glucose with a FPG of greater than equal to 100 to less than 126 mg/dL and/or impaired glucose tolerance with an OGTT of greater than or equal to 140 to less that 200 mg/dl. A person with Pre-Diabetes can have IFG and/or IGT in those ranges. In some embodiments, patients are selected that are overweight but not obese (have a BMI less than 30) and have Type 2 diabetes, that are overweight but not obese and have Pre-diabetes, or that have Type 2 diabetes and are not overweight or obese. In some embodiments, patients are selected that have one or more risk factors for Type 2 diabetes. These risk factors include age over 30, family history, overweight, cardiovascular disease, hypertension, elevated triglycerides, history of gestational diabetes, IFG, and/or IGT.

This disclosure includes systems and methods for treating impaired glucose regulation in a subject.

In embodiments, a method of treating a condition associated with impaired glucose regulation in a subject comprises applying an intermittent (or continuous) electrical signal to a target nerve of the subject, with the electrical signal selected to down-regulate neural activity on the nerve and to restore neural activity on the nerve upon discontinuance of the block. In some embodiments, the target nerve is the vagus nerve. In some embodiments, the site on the target nerve is located to avoid affecting heart rate such as below the vagal enervation of the heart. In some embodiments, the electrical signal is selected for frequency, amplitude, pulse width, and timing.

The electrical signal may also be further selected to improve glucose regulation. Improvement of glucose regulation can be determined by a change in any one of % of HbA1C, fasting glucose, or glucose tolerance test (IVGTT). In some embodiments, the method further comprises combining the application of an electrical signal treatment with administration of an agent that affects glucose regulation. In some embodiments, the application of the electrical signal treatment excludes application of an electrical signal treatment to other nerves or organs.

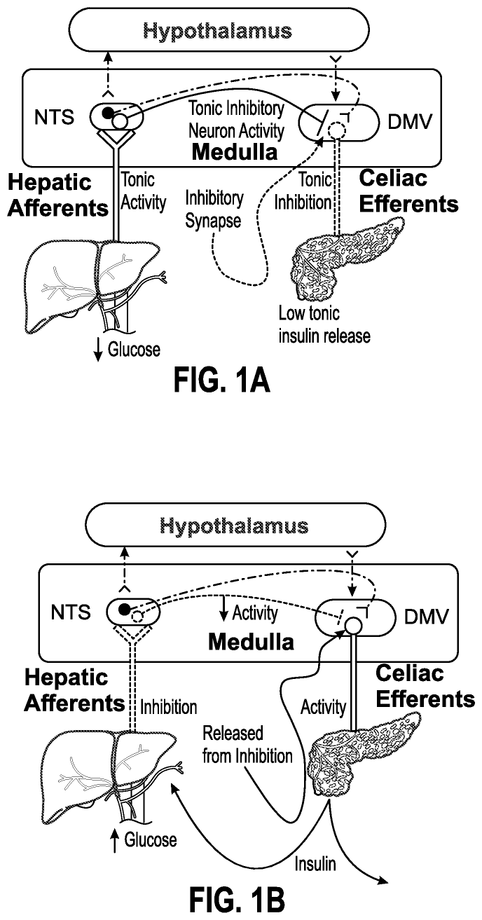

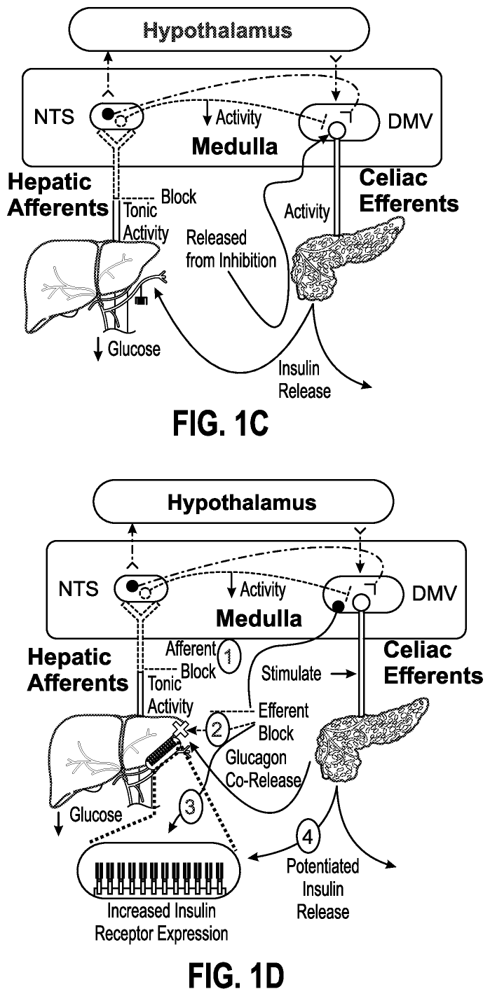

Referring now to FIG. 1, which depicts an example of example of an autonomic glycemic control reflex arc. Referring specifically to FIG. 1A which shows how tonic activity of vagus nerve's hepatic branch decreases pancreatic insulin release. An increase in hepatic branch activity excites inhibitory neurons in the nucleus tractus solitarius (NTS) in the brain stem. These neurons then project to the dorsal motor vagal (DMV) nuclei, also in the brain stem, resulting in decreased celiac branch efferent activity. Decreased celiac branch activity leads to decreased tonic insulin release. Referring now to FIG. 1B where portal vein glucose concentration induces a decrease in afferent hepatic axon activity, which leads to celiac axon activity upregulation. This celiac branch efferent activity will induce pancreatic insulin release. In some embodiments, it is desired to block conduction through the hepatic branch of the vagus nerve which inhibits hepatic neuron tone and, in turn, increase celiac efferent activity, as shown in FIG. 1C. In some embodiments a combination of vagal celiac branch stimulation, or alternatively stimulation at any location on the posterior vagus nerve cranial to the celiac branching point, with hepatic branch blockade can be utilized to influence various physiological processes leading to increased glycemic control (see FIG. 1D). In an alternative embodiment blockade at any location on the anterior vagus nerve cranial to the hepatic branching point can also be used to increase glycemic control. Referring now to FIG. 1D where the various mechanisms of how vagus branch hepatic block and vagus nerve celiac branch stimulation may increase glycemic control. In one example embodiment, the hepatic branch block decreases hepatic tone leading to increased tonic insulin release (see FIG. 1D-1). In another embodiment, the hepatic branch is blocked thereby decreasing the liver's sensitivity to glucagon (see FIG. ID-2). In yet another embodiment, the hepatic branch is blocked thereby increasing expression of hepatic insulin receptors (see FIG. 1D-3). In another embodiment, the celiac stimulation potentiating pancreatic insulin release (see FIG. 1D-4). It should be appreciated that any of the four aforementioned blocking protocols as shown in FIG. 1, could additionally include the step of delivering stimulation. In embodiments that include the additional stimulation step of the celiac branch can provide further glycemic control than block alone.

Referring still to FIG. 1, where FIG. 1(A) demonstrates how tonic activity of vagus nerve's hepatic branch decreases pancreatic insulin release. FIG. 1(B) demonstrates how increased portal vein glucose concentration induces a decrease in afferent hepatic axon activity which leads to celiac axon activity upregulation. FIG. 1(C) demonstrates how a block conduction through the hepatic branch of the vagus nerve inhibits hepatic neuron tone and, in turn, increases celiac efferent activity. FIG. 1(D) demonstrates a combination of vagal celiac branch stimulation with hepatic branch blockade to influence various physiological processes leading to increased glycemic control (FIG. 1(D)).

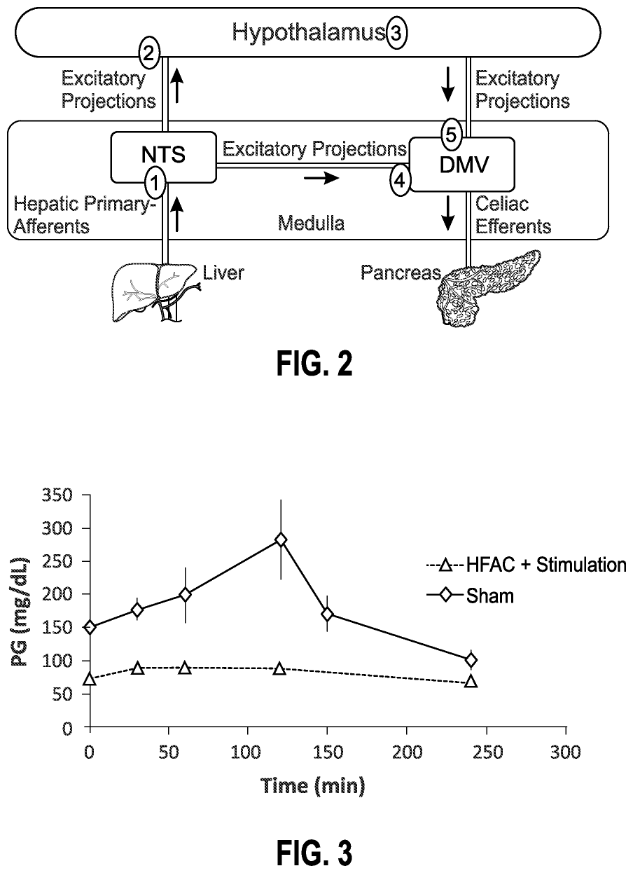

Referring now to FIG. 2, where there are 5 possible sites of neuronal plasticity in the glycemic regulation reflex arc 1) between hepatic afferents and their targets in the of the nucleus tractus solitarius (NTS), 2) between projection neurons from the NTS to the hypothalamus, 3) between intra-hypothalamic nuclei, 4) between excitatory synaptic projections between the NTS and DMV and 5) between projections from the hypothalamus to the NTS. It should be appreciated that plasticity can also occur in higher brain regions that are influenced by vagus nerve activity.

Referring now to FIG. 3, where HFAC delivered at the hepatic branching point with concurrent stimulation at the celiac branching point increased glycemic control during an OGTT compared to sham. The HFAC and stimulation signals were delivered continuously during the 4 hour OGTT experiments. As shown plasma glucose levels were kept to levels below 100 mg/dl from 0 to 250 minutes, whereas the sham group included steady increase between 0 and 125 minutes to above 250 m/dL and returned to slightly above 100 mg/dL between 125 and 250 hours.

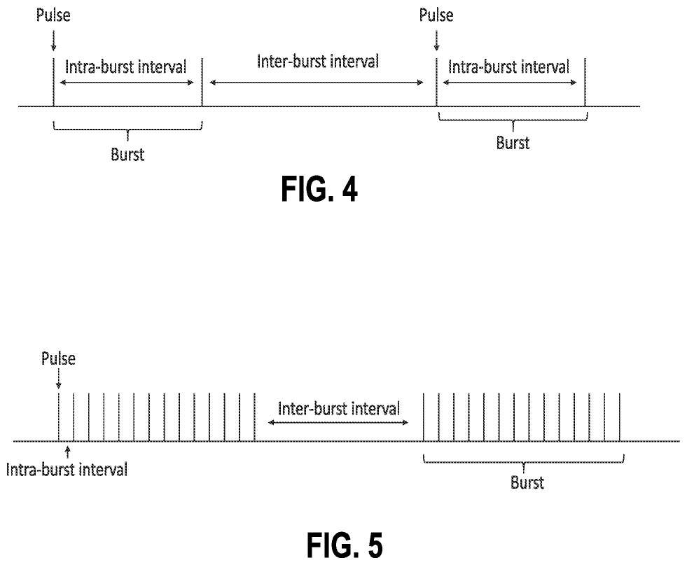

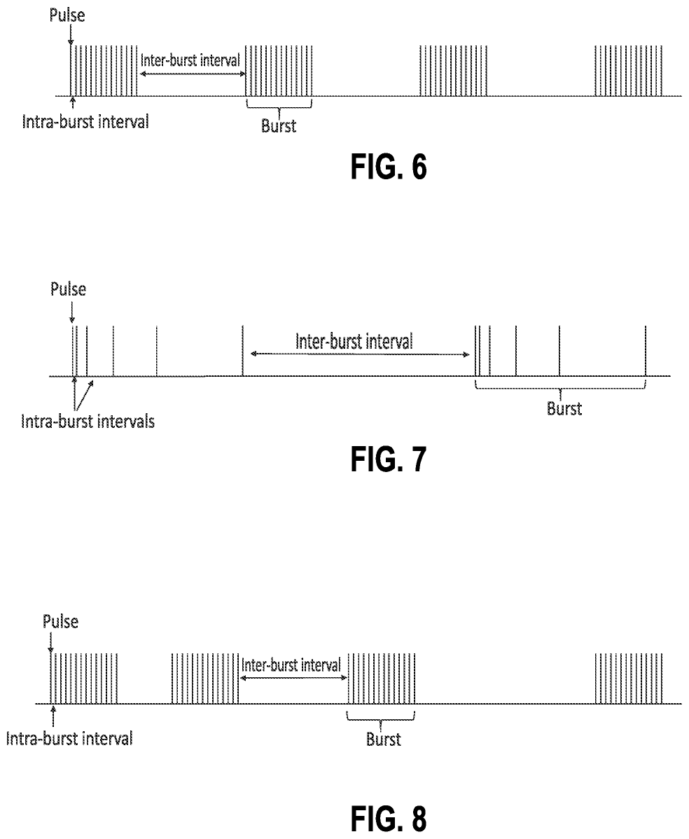

Referring now to FIG. 4, where one protocol which comprise a burst stimulation with 2 pulses in one burst. Referring now to FIG. 5, where the burst stimulation protocol includes multiple pulses in one burst and two bursts. Referring now to FIG. 6 where the burst stimulation comprises multiple pulses and multiple bursts. FIG. 7 includes burst stimulation with non-fixed intra-burst intervals. Referring now to FIG. 8, where burst stimulation comprises delivery of non-fixed inter-burst intervals.

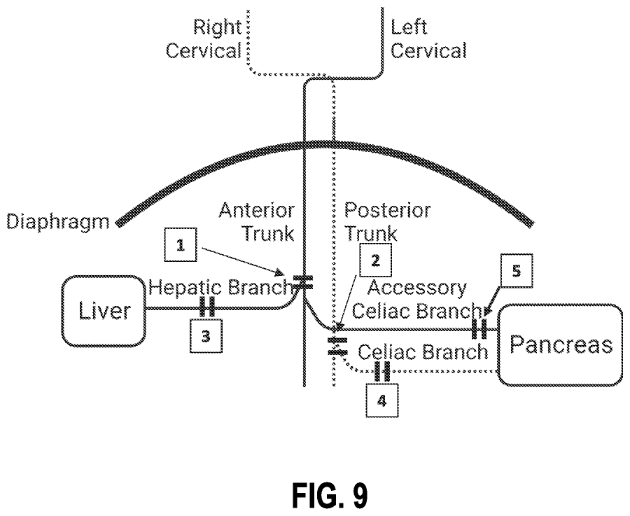

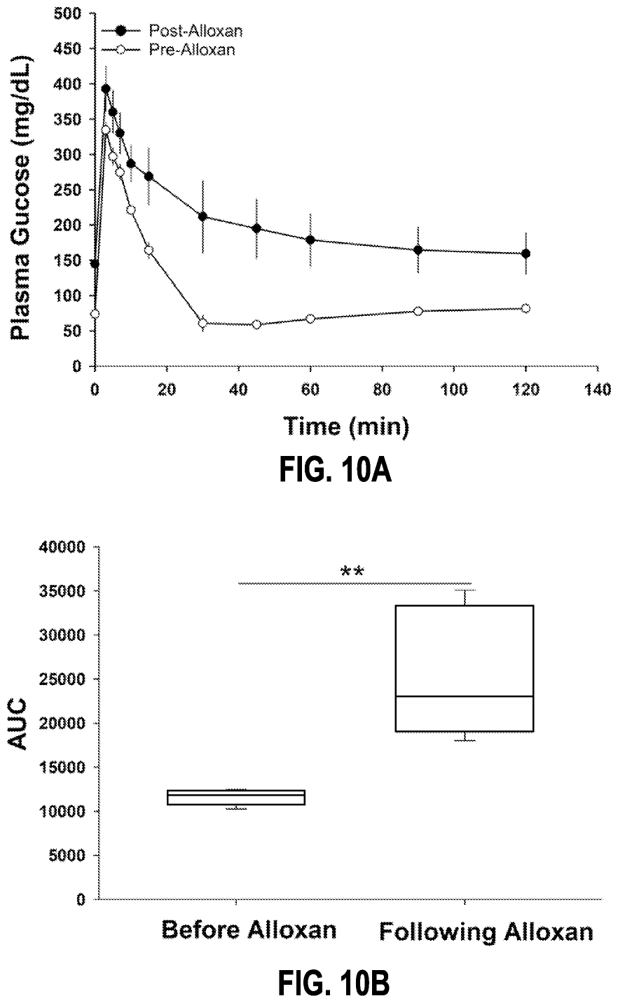

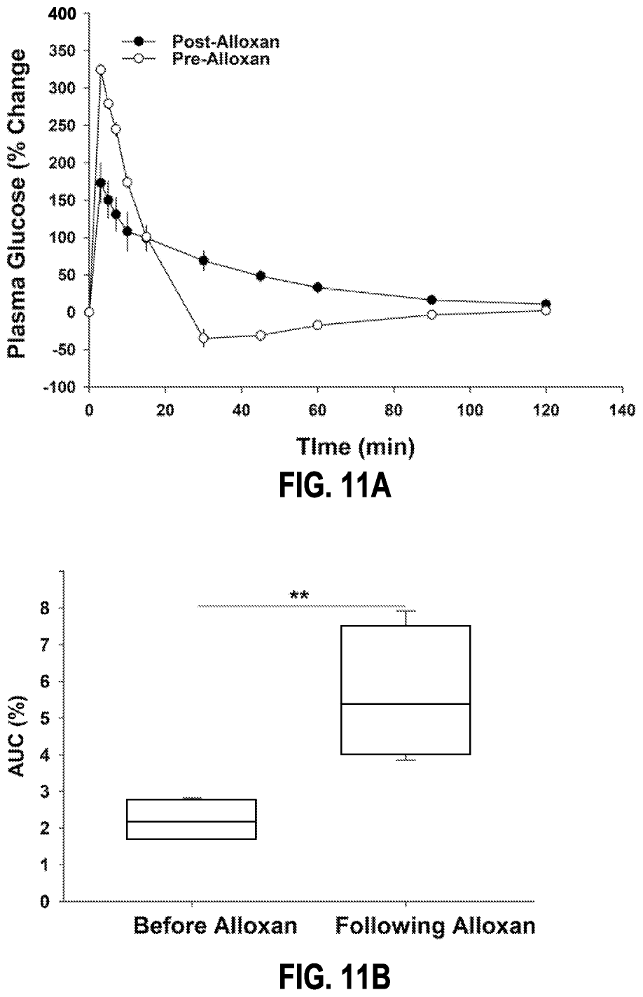

Referring now to FIG. 9, where a diagram illustration shows the various sites of neuromodulation of the vagus nerve. Site 4 and 5 include similar effects. Referring now to FIG. 10, where IVGTT in swine pre-and post-alloxan treatments with absolute values of plasma glucose in mg/dL (FIG. 10A). FIG. 10B is a bar graph where the analysis of FIG. 10A where area under the cure of the graphs in ** p<0.05, Mann Whitney U test. Referring now to FIG. 11 where the same IVGTT protocol is performed as in FIG. 10, where the pre-and post-alloxan treatments with % change in plasma glucose. FIG. 11B is a bar graph with analysis of the experiments performed in FIG. 11A, analysis is indicated where ** is shown with a p-value of p<0.05, using the Mann-Whitney U test.

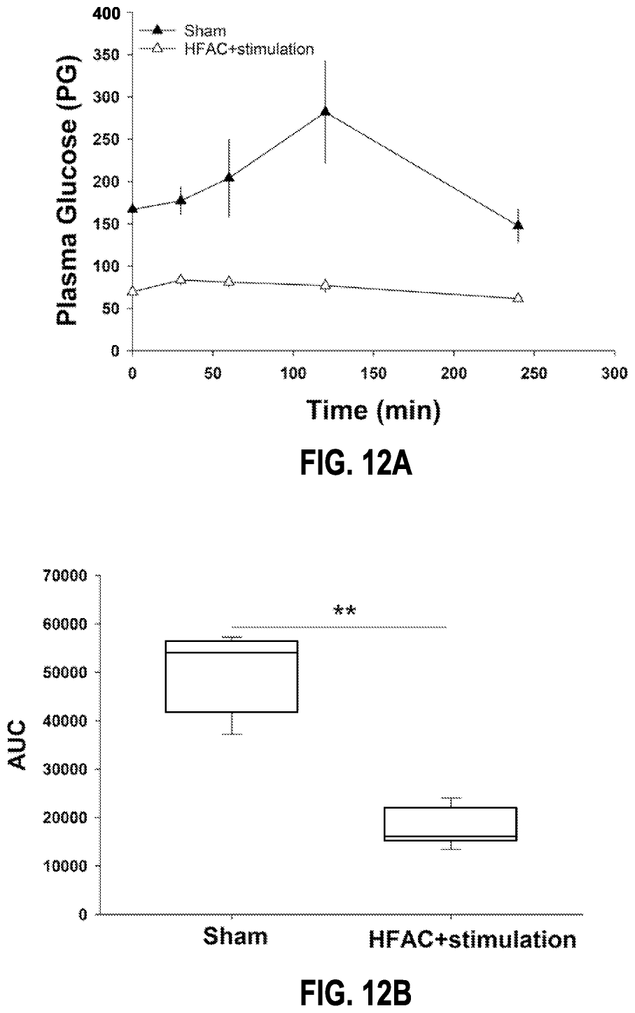

Referring now to FIG. 12A where the HFAC delivered at the hepatic branching point using concurrent stimulation at the celiac branching point increased glycemic control during an OGTT compared to sham. Data is in absolute plasma glucose levels (mg/dL) The HFAC and stimulation signals were delivered continuously during the 4-hour OGTT experiments. FIG. 12B is the graphical analysis of the experiments in FIG. 12A using the Area under the cure. where analysis is performed with a p-value of ** p<0.05, Mann-Whitney U test.

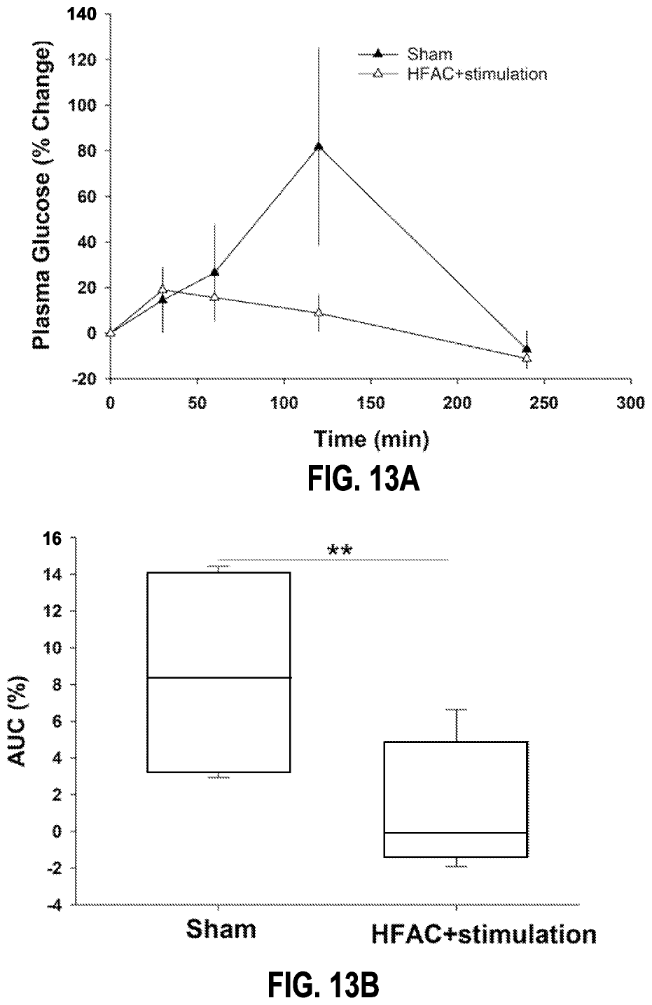

Referring now to FIG. 13 where HFAC delivered at the hepatic branching point with concurrent stimulation at the celiac branching point increased glycemic control during an OGTT compared to sham. This is the same experiment as in FIG. 12 except data is in % change from baseline. The HFAC and stimulation signals were delivered continuously during the 4-hour OGTT experiments. FIG. 13B provides analysis of the data in FIG. 13A using AUC of the graphs in a. ** p<0.05, Mann-Whitney U test.

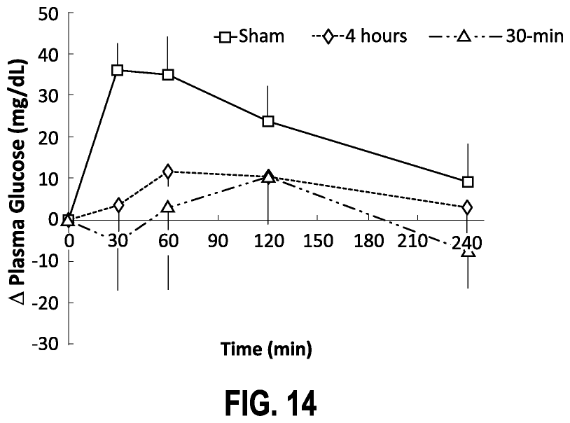

Referring now to FIG. 14 where HFAC delivered at the hepatic branching point with concurrent stimulation at the celiac branching point increased glycemic control during an OGTT with signals delivered continuously during the 4-hour OGTT or intermittently during the first 30 minutes of the OGTT.

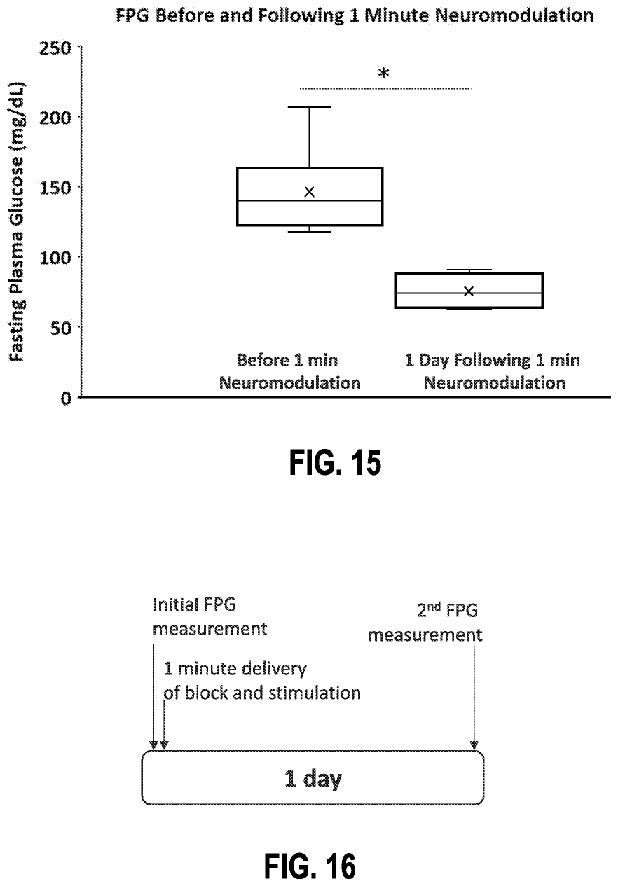

Referring to FIG. 15 where FPG before 1 min of HFAC delivered at the hepatic branching point with concurrent stimulation at the celiac branching and 1 day after the termination of the signals. Statistical analysis includes a p-value of *p=0.00092, Students t-test, Data are normally distributed, Shapiro-Wilk Test. Sample included n value of 4, using Box Plot, Whiskers=Minimum and Maximum, Box is 1st and 3rd Quartiles, Line=Median and x=Mean.

Referring to FIG. 16 which is an illustration of time course performed during the experiments in FIG. 15.

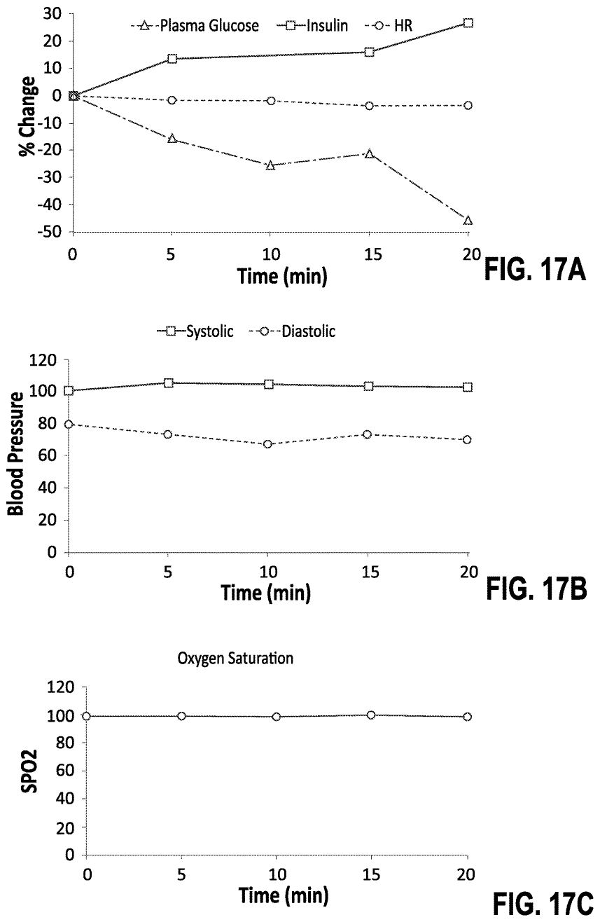

FIG. 17A is a % change in plasma glucose, insulin, and heart rate (HR) during 5 Hz stimulation of the celiac branch in swine. FIG. 17B shows the changes in blood pressure during delivery of 5 stimulation of the celiac branch in swine. FIG. 17C indicates oxygen saturation during delivery of 5 Hz stimulation of the celiac branch.

It should be appreciated that the changes in the synapses of the nerves being delivered a signal (block and/or stimulation) are plastic. This phenomenon is based on the activation of a presynaptic neuron induces either long-term potentiation (LTP) or long-term depression (LTD) of the synapse between the pre-and postsynaptic neuron. The pattern of activity of the presynaptic neuron induces either LTP or LTD.

LTP induces a larger postsynaptic depolarization to an incoming presynaptic release of neurotransmitter. The larger postsynaptic depolarization will increase the probability of a generation of an action potential by the postsynaptic neuron. LTD has the opposite effect of LTP. Following LTD a smaller postsynaptic depolarization to an incoming presynaptic release of neurotransmitter occurs. This decreases the probability of the generation of an action potential by the postsynaptic neuron. The majority, if not all, of LTP and LTD events occur at glutamatergic synapses.

There are multiple plastic glutamatergic synapses in the glycemic reflex arc that offer many opportunities for activity-induced, long-lasing changes to increase glycemic control. FIG. 2 label 5 of such synapses: 1) between hepatic afferents and their targets in the of the nucleus tractus solitarius (NTS), 2) between projection neurons from the NTS to the hypothalamus, 3) between intra-hypothalamic nuclei, 4) between excitatory synaptic projections between the NTS and DMV and 5) between projections from the hypothalamus to the NTS. It is important to note that plasticity can also occur in higher brain regions that are influenced by vagus nerve activity.

It is the scope of this present disclosure to modulate a peripheral nerve, such as the vagus nerve, in a manner that causes a plastic change in the glycemic regulatory system, with or without the involvement of the central nervous system (CNS). These changes in plasticity effect the activity of an organ. Plasticity as defined within the scope of the present disclosure is defined as a change in a physiological process that outlasts the termination of an applied electrical signal. Plasticity may occur at the level of the CNS or at a peripheral organ(s), not involving the CNS, that are involved in glycemic control such as (but not limited to) the liver, pancreas, duodenum, skeletal muscle or adipose tissue or combination thereof. This may be achieved through either short duration low frequency nerve stimulation or application of high frequency alternating current (HFAC) or through burst stimulation or any combination thereof.

Current amplitudes of 0.1 mA to 20 mA for the HFAC signal, the low frequency stimulation signal or burst stimulation signal may be desired. Pulse widths for the low frequency stimulation signal or burst stimulation signal may be from 0.01 ms to 10 ms. The HFAC signal may be a frequency of 200 Hz and above, more specifically 500 Hz to 5 kHz, 1 khz to 10 kHz and 10 kHz to about 80 kHz. The frequency of the low frequency stimulation signal may be from 0.01 Hz to 199 Hz. Or 0.1 to 10 Hz, or 10 Hz to 30 Hz or 30 Hz to 100 Hz or from 100 Hz to 199 Hz.

The ability to induce long-lasting positive effects on glycemic control (such as decreased fasting plasma glucose (FPG), decreased HbA1c, decreased amplitude of plasma glucose spikes throughout the day or decreased variability of the amplitude of plasma glucose spikes throughout the day, as measured by the standard deviation (below one third of mean glucose) or coefficient of variation (below 33% variation) of plasma glucose) with a minimal amount of energy is desirable to create a small implantable pulse generator or an implantable generator that needs infrequent charging (once per week to once per 12 months), an implantable pulse generator that is a primary cell device (no need for charging for the life of the device (typically from 3-5 years or 5-10 years), or any combination of these properties. It should be appreciated that in some example embodiments the small implantable generator will have a length of no greater than 1.5 inches. In other related embodiments, the total volume of the implantable pulse generator will not exceed 0.5 in.³, 1 in.³, 1.5 in.³, 1.75 in.³, or 2.0 in.³. The small size of the implantable pulse generator is such to deliver an electrical signal to the target nerve, while also requiring reduced energy to perform the function, thereby allowing for long use periods without the need for recharging. Moreover, in embodiments where the implantable pulse generator requires a recharge, the amount of time to recharge the implantable pulse generator is also reduced compared to currently available pulse generators.

The amount of gained energy efficiency by only requiring short duration HFAC (such as 1 minute) compared to a prolonged HFAC application. This can be shown for example, in FIG. 3, where an increase glycemic control can be achieved when applied to the sub-diaphragmatic vagus nerve branches and paired with low frequency stimulation (see oral glucose tolerance test in alloxan treated swine). It should be appreciated that using the present disclosure a calculation total charge to be delivered is performed by assessing the area under the curve of the pulses during their delivery. For example, under 4 hours of application of a 5 kHz HFAC 8 mA 90 μsec pulse width signal is 0.000090 sec (pulse width)×5,000 (pulses/second)×14,400 seconds (4 hrs of signal delivery)×0.008 (Amps amplitude of the signal)=51.84 coulombs. For 1 minute of 5 k Hz application with the same parameters the total delivered charge would be 0.000090 sec (pulse width)×5,000 (pulses/second)×60 (seconds 1 minute signal delivery)×0.008 (Amps)=0.216 coulombs. This equates to 96% less charge delivered of the 1-minute 5 kHz signal compared to the 4-hr signal. In other alternative embodiments the charge using the short duration protocol is delivered using: at least 60% less charge, at least 65% charge, at least 70% charge, at least 75% charge, at least 80% less charge, at least 85% charge or at least 90% charge.

The amount of gained energy efficiency by only requiring 1 minute of a low frequency signal compared to a 4 hour low frequency application (which has previously been shown to increase glycemic control when applied to the sub-diaphragmatic vagus nerve branches and paired with a HFAC signal (as shown through an oral glucose tolerance test in alloxan treated swine) when paired with a high frequency signal in our studies (FIG. 3)) can be appreciated by calculating the total charge delivered (which is the area under the curve of the pulses during their delivery). For 4 hours of application of a 1 Hz 8 mA 4 millisecond pulse width signal is 0.004 sec (pulse width)×1 (pulses/second)×14,400 seconds (for 4 hrs of signal delivery)×0.008 (Amps)=0.4608 coulombs. For 1 minute of application with the same parameters would be 0.004 sec (pulse width)×1 (pulses/second)×60 (seconds 1 minute signal delivery)×0.008 (Amps)=0.00192 coulombs. This equates to 96% less charge delivered of the 1-minute 1 Hz signal compared to the 4-hr signal. In other alternative embodiments the charge using the low frequency protocol is delivered using: at least 60% less charge, at least 65% charge, at least 70% charge, at least 75% charge, at least 80% less charge, at least 85% charge or at least 90% charge.

The combined total charge of the delivery of the HFAC and 1 Hz signal for 4 hrs=51.84 coulombs+0.4608 coulombs=52.3008 coulombs. The combined total charge of the delivery of the HFAC and 1 Hz signal for 1-minute=0.216 coulombs+0.00192 coulombs=0.21792 coulombs. This equates to 96% less charge delivered of the 1-minute 5 kHz and 1 Hz signal compared to the 4-hr 5 kHz and 1 Hz signal.

Lasting effects of enhanced glycemic control with a 1-minute delivery of 5 kHz and 1 Hz to the sub-diaphragmatic vagal trunks is an example of plasticity in the glycemic regulatory system and this plasticity may be used as a method to substantially increase neuromodulation efficiency that improves glycemic control to a patient in need thereof. Plasticity can occur through neuronal or extra-neuronal activity where examples of extra-neuronal activity would be physiological processes within a visceral organ or between visceral organs that regulate plasma glucose concentration. Examples of organs that regulate plasma glucose concentration are the liver or pancreas or duodenum or skeletal muscle or adipose tissue. Plastic changes can occur in one or multiple organs.

The electrical signal can be characterized by multiple stimulation waveforms pulses or by total charge delivered during the application of the electrical signal. Waveforms pulses can consist of a fixed frequency of pulses or a burst of pulses. A burst of pulses consists of at least 2 pulses followed by delivery of at least another 2 pulses separated by an inter-burst interval (see FIG. 4).

As shown in FIG. 4, the inter-burst interval is greater than the time separating the 2 or more pulses. The time separating the 2 or more pulses is termed the intra-burst interval. The intra-burst interval is less than the inter-burst interval. A burst with multiple pulses is depicted in FIG. 5. An example of burst stimulation with multiple pulses and multiple bursts is depicted in FIG. 6. A burst stimulation may not have fixed intra-burst intervals (see FIG. 7). A burst stimulation may not have fixed inter-burst intervals (FIG. 8). The frequency of intra-burst intervals can range from 1 to 199 Hz. The inter-burst interval can range from 1 second to 20 seconds. The duration of burst stimulation can last from about 10 seconds to about 30 minutes.

The electrical signal can be delivered at a fixed continuous frequency. The frequency can range from 0.01 Hz to 199 Hz. The duration of the application of the electrical signal delivered at a fixed frequency can last from about 1 second to about 1 hour.

The waveforms characteristics may consist of square mono-phasic or square bi-phasic pulses or sinusoidal or triangular or sawtooth pulses with base to peak amplitudes of 0.01 mA to 20 mA. Square waveform pulse width may range from 0.01 milliseconds to 20 milliseconds.

The electrical signal may be characterized by the total charge delivered regardless of waveform characteristics or pulse delivery pattern (bursting or a fixed continuous frequency application). Charge ranges include 1.25×10⁻⁶ to 286 coulombs, 2.5×10⁻⁵ to 0.288 coulombs, 3.2×10⁻³ to 0.16 coulombs, or about 1.28×10⁻² coulombs, 9.38×10⁻⁵ to 0.144 coulombs, 9.6×10⁻³ to 4.8×10⁻² coulombs, or about 1.2×10\hat{ }−2 coulombs.

Delivery of bursting or fixed frequency electrical signals can occur once or multiple times in one day or once per week or once per month.

### EXAMPLES

### Example 1

### Block (@5000 Hz) and Stimulation (@1 Hz) Increased Glycemic Control in a Zucker Rat Model of Type 2 Diabetes

Experiments were conducted on a well-established Zucker rat model of type 2 diabetes. The experiment consisted of 5 conditions: a sham operation (n-6), a vagotomy+stimulation (@ 1 Hz, vagotomy at site 3 on FIG. 9, stimulation at site 4 on FIG. 9) (n=4), block (@ 5000 Hz, at site 3 on FIG. 9) of the hepatic vagal branch with concurrent stimulation (@ 1 Hz, site 4 on FIG. 9) of the vagal celiac branch (n=5), hepatic vagotomy alone (site 3 on FIG. 9, n=4) and celiac stimulation (site 4 on FIG. 9, @ 1 Hz) alone (n=4). The glucose response was quantified by using an established method of calculating the area under the curve (AUC, area unit=glucose*time=AU) of intravenous glucose tolerance tests (IVGTTs). Comparisons between the condition tested and sham consisted of a student's t-test and an alpha level of ≤0.5 as considered significant and data are presented as mean±SEM.

To decrease the effective variability in fasting plasma glucose (FPG, range about 200-300 mg/dL) between animals, changes in glucose were normalized to baseline glucose. One hour following these procedures an IVGTT was administered as described by Nagase et al, the disclosure incorporated herein in its entirety.

In the block and stimulation, which was applied 15 minutes prior to and during the entire course of the 30-minute IVGTT, and vagotomy+stimulation groups there was a significant sustained decrease in AUC compared to sham (sham=1543±257 AU, vagotomy+stimulation=618±111 AUC p<0.01, block and stimulation=898±68 AUC p<0.05). For the hepatic vagotomy alone and the stimulation alone groups there was no significant difference in AUC compared to sham following the challenge. Fifteen min following cessation of block and stimulation a second glucose injection induced a large increase in plasma glucose AUC which was non-significant, to a subsequent glucose injection in the sham group (sham second injection AUC=2603±310 AU, block and stimulation second injection AUC=1820±301 AU, p=0.15). This suggests a functional recovery, i.e., the rats demonstrated glucose intolerance following the cessation of block and stimulation. Also, the increase in glycemic control in the block and stimulation group was similar to the vagotomy+stimulation group demonstrating that 5000 Hz mimicked the vagotomy suggesting a reversible electrical conduction block.

While not wanting to be bound by any particular theory, the mechanism contributing to the effect of block and stimulation in these experiments may include by are not limited to decreased insulin resistance, a key hallmark of type 2 diabetes. Second, hepatic block causing a decrease in expression of PPARa. Third, release of celiac afferent axons from inhibition leading to increased insulin release. Fourth, decreased hepatic sensitivity to glucagon via hepatic block. Fifth, insulin release due to celiac stimulation. It should be appreciated that any 1 or combination of the aforementioned mechanisms can be utilized to get an effective block and/or stimulation. Regardless of mechanisms, it appears that the combination of stimulation and block is superior to standalone conditions on increasing glycemic control, the ultimate goal for T2DM treatments.

### Example 2

### Electrophysiology Experiments in Isolated Swine Sub-Diaphragmatic Vagus Nerve

In isolated sub-diaphragmatic electrophysiology stimulation experiments (n=3), the swine vagus nerve was suspended on 2 electrodes in oil. One bipolar electrode was used for stimulation (a biphasic square wave) and the other for recording compound action potentials (CAPs conduction velocities<1 m/s). The nerve was stimulated at 1 Hz and the anode contact was closest to the recording electrode. A strength-duration curve was then constructed w/Chronaxie=1.27±0.24 m/s and Rheobase=3.67±0.72 mA. A current amplitude of 8 mA with a pulse width of 4 ms maximized the amplitude of the CAP and this combination was used in the swine experiments to demonstrate proof of concept and safety.

Experiments conducting block (HFAC @5000 Hz) on isolated swine sub-diaphragmatic vagus nerve blocking experiments (n=6) in which the vagus nerve was suspended on 4 electrodes in oil. The electrodes consisted of, in order, a distal stimulation electrode, an electrode delivering 5000 Hz HFAC, a proximal control electrode to test for stimulation and a recording electrode. CAPs (conduction velocity<1 m/s) were detected by the recording electrode by stimulation of the distal and proximal electrodes prior to and within 1 sec following the cessation of 1 min of 5000 Hz.

A decrease in the CAP amplitude with proximal stimulation indicates 5000 Hz-induced stimulation and a decrease in the CAP with distal stimulation indicates a 5000 Hz-induced conduction block. At 8 mA there was little to no stimulation and a full conduction block was achieved and this amplitude was used in in vivo swine experiments. It should be noted that block may occur at different current amplitudes than 8 mA depending on electrode geometry, electrode to nerve contact and impedance.

### Example 3

### Addition of Stimulation to Block was Superior to Block Alone in Alloxan Treated Swine

There are mixed reports about whether hepatic vagotomy increases glycemic control. This leads to the question whether the HFAC-induced block (@5000 Hz) is sufficient to increase glycemic control or if there is necessity of the addition of stimulation to increase glycemic control. To address this, we evaluated if HFAC in a swine model of type 2 diabetes. The animal model of type 2 diabetes was created by alloxan treatment in Yucatan swine. Following alloxan the swine became glucose intolerant indicated by decreased performance on IVGTTs (FIG. 10A, 10B, 11A and 11B), but the swine were not insulin dependent, indicative of a type 2 diabetic state. Oral glucose tolerance tests (OGTTs) were utilized to access glycemic control. The OGTTs consisted of oral consumption of 75 g of glucose dissolved in 100 mL of diet Gatorade. We found that there was no difference in glycemic control between sham and hepatic branch HFAC block alone (block was applied to site 1 in FIG. 9). There was a significant increase in glycemic control with block and stimulation (stimulation location is indicated at site 2 in FIG. 9) compared to sham and block alone. Neuromodulation to enhance glycemic control may not be limited to combined block and stimulation. This example serves to demonstrate that neuromodulation of sub-diaphragmatic vagal trunks and branches holds the ability to influence glycemic control and that stimulation may enhance the effects of block under these conditions.

### Example 4

### Safety on Organ Systems Innervated by the Celiac and Hepatic Branches of the Vagus Nerve was Successfully Demonstrated Following Block and Stimulation Experiments

Following 1 month of block and stimulation application in alloxan treated type 2 diabetic swine, sections of the brain, liver and pancreas were prepared for histopathologic analysis. No damage to the vagus nerve at the sites of electrodes delivering TDN was observed. Sections of brain were considered to be within normal limits. There are clear areas surrounding many of the cells and vasculature in the sections, which has been reported as a processing artifact in neurologic tissue. Edema cannot be completely excluded but is considered less likely given the absence of any other overt pathologic changes. Sections of liver showed mild changes (hydropic change, biliary hyperplasia) that are likely to be alloxan induced model-associated. Two if the four animals had increases in gamma-glutamyltransferase (GGT) over the course of the study, which can be seen with biliary hyperplasia. These increases are very mild and unlikely to have clinical relevance. There was also a gross and histopathologic fibrous connective tissue response associated with the device leads. This is an expected response to foreign material and is unlikely to be of clinical relevance. The most notable findings in this study were present in the histology of pancreatic tissue. Many of the changes are likely to be alloxan-model-related in islet cell atrophy, vacuolization, necrosis, and apoptosis, which have been reported in animals treated with alloxan. Similar findings of islet atrophy, vacuolization, necrosis, and apoptosis were observed in a control alloxan treated swine pancreas which did not receive block and stimulation signals. Overall, tissues of interest were healthy or had mild changes, which are unlikely to have clinical relevance.

### Example 5

### One Minute Block and Stimulation Experiment Demonstrating Plasticity of the Glycemic Control with Sub-Diaphragmatic Neuromodulation in Type 2 Diabetic Alloxan Treated Swine

Prior example experiments demonstrated that delivery of continuous HFAC (@5000 Hz, 8 mA,) to the sub-diaphragmatic vagal hepatic branching point of the vagus nerve (site of 5000 Hz block indicated at site 1 on FIG. 9) with concurrent stimulation (@1 Hz, 8 mA, 4 ms pulse width,) to the sub-diaphragmatic vagal celiac branching point of the vagus nerve (site of stimulation indicated at site 2 on FIG. 9) increases glycemic control during the duration of 4 hour OGTTs in alloxan treated swine (FIG. 12A, 12B, 13A and 13B). It has also been demonstrated that delivery of continuous HFAC (@5000 Hz, 8 mA) to the sub-diaphragmatic vagal hepatic branching point of the vagus nerve with concurrent stimulation (@1 Hz, 8 mA, 4 ms pulse width) to the sub-diaphragmatic vagal celiac branching point of the vagus nerve during only the first 30 min of 4-hour OGTTs increases glycemic control in alloxan treated swine (FIG. 14). This demonstrates that an intermitted delivery of block (@5000 Hz) and stimulation (@1 Hz) signals delivered to the sub-diaphragmatic vagus nerve can blunt a glucose spike without prolonged signal application. For example, a 30 min delivery time compared to 4-hour delivery time. This intermittent delivery decreases power consumption by an eighth. Further experiments were conducted to determine if shorter durations of block and stimulation can increase glycemic control.

One minute delivery of HFAC (@5000 Hz, 8 mA) to the sub-diaphragmatic vagal hepatic branching point of the vagus nerve (site 1 on FIG. 9) with concurrent stimulation (@1 Hz, 8 mA, 4 ms pulse width) to the sub-diaphragmatic vagal celiac branching point of the vagus nerve (site 2 on FIG. 9) significantly increased glycemic control by observation of a decrease in fasting plasma glucose (FPG). Fasting plasma glucose prior to the one-minute signals was 142±12 mg/dL (range 117 to 207 mg/dL) which significantly decreased to 76±6 mg/dL (range 63 to 91 mg/dL, FIG. 15, p=0.00092, students t-test, n=4 swine and data was normally distributed demonstrated by Shapiro-Wilk analysis) following 1 day after termination of the signals (see timeline depicted in FIG. 16). These results indicate two differentiating factors compared to prior experimental data, as well as differentiating neuromodulation site location that is typically performed in the field of vagus nerve stimulation for the treatment of disease, such as epilepsy and depression. The prolonged increase in glycemic control following the termination of block and stimulation demonstrates neuromodulation-induced plasticity of the glycemic regulatory system with only 1 minute of application (vs 30 minutes or 4 hours perform in previous experiments). This short duration of neuromodulation is typically used to induce synaptic plasticity with bursting or fixed continuous frequency.

The other differentiating feature of the present disclosure is lowering off-target effects compared to established vagus nerve stimulation paradigms. Clinically, vagus nerve stimulation is used for the treatment of epilepsy and refractory depression with stimulation localized to cervical segments of the vagus nerve. This stimulation site causes unwanted off-target effects such as effects on speech due to stimulation of laryngeal muscles due to stray currents, changes in heart rate, blood pressure and respiration. Vagal neuromodulation that limits (or negates) these off target effects is desirable. In one set of experiments, we tested the hypothesis that sub-diaphragmatic vagal nerve stimulation with our system can influence pancreatic activity and changes in plasma glucose without inducing changes in heart rate, blood pressure and respiration. In these experiments, 5 Hz stimulation was applied to the celiac branch of the vagus nerve in swine (site of stimulation depicted in FIG. 9 at location site 4). This induced a decrease in plasma glucose and an increase in plasma insulin without causing a change in heart rate (FIG. 17A). Furthermore, a change in blood pressure or respiration (as assessed changes in oxygen saturation) were not observed during sub-diaphragmatic stimulation (FIG. 17B and 17C). Finally, since the stimulation did not occur at the cervical location, there would not be an expected stimulation of laryngeal muscles due to distance from these muscles. Having the ability to influence glycemia via vagus nerve neuromodulation without off-target cardiac, blood pressure or respiratory effects. While also reducing or eliminating speech effects allows for greater flexibility in selecting stimulation parameters to induce desired effects on glycemia. These stimulation parameters include pulse amplitude, pulse width, duration of application of neuromodulation and type of neuromodulation such a bursting or fixed continuous frequency applications.

Other Diseases that could be treated by inducing plasticity with sub-diaphragmatic stimulation may be (but not limited to) obesity, diseases caused by inflammation, autoimmune diseases, rejection of organ transplant, pancreatitis, hypertension and cancer.

Although the present disclosure has been described with respect to one or more particular embodiments, it will be understood that other embodiments of the present disclosure may be made without departing from the scope of the present disclosure.

Further examples and embodiments of the present disclosure are disclosed in the enumerated clauses which follow:
1. A system for altering plasticity in a target comprising:
at least two electrodes operably connected to an implantable pulse generator, wherein at least one of the electrodes is adapted to be placed on a target to alter the plasticity.
2. The system of clause 1, wherein the implantable pulse generator comprises a power module and a programmable therapy delivery module, wherein the programmable therapy delivery module is configured to deliver at least one therapy program comprising an electrical signal treatment applied to the target, wherein the electrical signal has a frequency selected to initiate activity on the target; and
an external component comprising a communication system and a programmable storage and communication module, wherein programmable storage and communication module is configured to store the at least one therapy program and to communicate the at least one therapy program to the implantable pulse generator and wherein the activity is a neural stimulation or a neural block.
3. The system of clause 2, wherein the electrical signal treatment is continuously applied to the target nerve.
4. The system of clause 2, wherein the electrical signal treatment is bursting neuromodulation applied to the target.
5. The system of any one of clauses 1-4, wherein the electrical signal has an on time and an off time, wherein the off time is selected to allow at least a partial recovery of the activity of the target nerve.
6. The system of any one of clauses 1-5, wherein the off time is configured to commence upon the detection of blood glucose levels between 80 mg/dL and 110 mg/dL.
7. The system of any one of clauses 1-6, where the communication system is selected from a group consisting of an antenna, blue tooth technology, radio frequency, WIFI, light, sound and combinations thereof.
8. The system of any one of clauses 1-7, wherein the at least one electrode is adapted to be placed on an organ selected from the spleen, stomach, duodenum, pancreas, liver and ileum.
9 The system of any one of clauses 1-8, wherein the at least one electrode is adapted to be placed at a target, wherein the target is a nerve selected from a group comprising a vagus nerve, a splanchnic nerve, a hepatic branch of a vagus nerve, a celiac branch of a vagus nerve and combinations thereof.
10. The system of any one of clauses 1-9, wherein the programmable therapy delivery module is configured to deliver an electrical signal having a frequency of at least 200 Hz.
11. The system of any one of clauses 1-9, wherein the programmable therapy delivery module is configured to deliver an electrical signal having a frequency of 500 Hz to 5 kHz, 1 kHz to 10 kHz or 10 kHz to about 80 kHz.
12. The system of any one of clauses 1-9, wherein the programmable therapy delivery module is configured to deliver a HFAC, low frequency stimulation or burst stimulation signal.
13. The system of clause 12, wherein current amplitudes of HFAC, low frequency stimulation or burst stimulation signal is between 0.1 mA to 20 mA.
14. The system of any one of clauses 12-13, wherein a pulse width for the low frequency stimulation signal or burst stimulation signal is between 0.01 ms to 10 ms.
15. The system of any one of clauses 12-13, wherein the frequency of the low frequency stimulation signal is from 0.01 Hz to 199 Hz, 0.1 to 10 Hz, 10 Hz to 30 Hz, 30 Hz to 100 Hz, 100 Hz to 199 Hz.
16. The system of any one of clauses 1-15, wherein the total volume of the implantable pulse generator will not exceed 0.5 in.³, 1 in.³, 1.5 in.³, 1.75 in.³, or 2.0 in.³.
17. The system of any one of clauses 1-16, wherein the combined total charge of the delivery of the HFAC and 1 Hz signal for 1-minute is less than 40 coulombs.
18. The system of any one of clauses 1-16, wherein the delivery of a 1 minute 5 kHz signal is at least 90% less than the same 5 kHz signal delivered for 4 hrs.
19. The system of any one of clauses 1-18, wherein the electrical signal is delivered between 1 second to 1 hour.
20. The system of any one of clauses 12-18, wherein the electrical signal is a burst signal and the burst signal is delivered between about 10 seconds to about 30 minutes.
21. The system of any one of clauses 1-20, wherein the system for inducing plasticity is delivered to a sub-diaphragmatic target.
22. The system of any one of clauses 1-21, wherein the system treats a diseases selected from a group comprising obesity, inflammation, autoimmune diseases, rejection of organ transplant, pancreatitis, hypertension, cancer and any combination thereof.
23. The system of any of clauses 1-22, wherein the programmable therapy delivery module is configured to deliver an electrical signal having an off time of at least 30 minutes between a second electrical treatment application.
24. The system of any of clauses 1-23, wherein the programmable storage and communication module is configured to store and communicate more than one therapy program, wherein each therapy program is different from one another, and is configured to be selected for communication.
25. The system of any of clauses 1-24, wherein programmable therapy delivery module is configured to deliver a second therapy program comprising an electrical signal treatment applied to a second target, wherein the second target is a nerve or an organ.
26. The system of any of clauses 1-25, wherein the second target nerve is the splanchnic nerve or the celiac branch of the vagus nerve, or the dorsal vagus nerve central to the branching point of the celiac nerve.
27 A method of for altering plasticity in a target comprising:
applying an electrical signal to a target nerve or organ using the system of any one of clauses 1-26.
28. A method of for altering plasticity in a target comprising: applying an electrical signal to a target nerve or organ of the subject having impaired glucose regulation using the system of any one of clauses 1-26.
29. A method of for altering plasticity in a target comprising:
applying an electrical signal to a target nerve or organ of the subject having type-2 diabetes using the system of any one of clauses 1-26.
30. The method of any one of clauses 27-29, further comprising administering an agent that improves glucose control.
31. The method of clause 30, wherein the agent increases the amount of insulin and/or increases the sensitivity of cells to insulin.
32. The method of clause 31, wherein the agent that increases the amount of insulin is selected from the group consisting of insulin, insulin analogs, sulfonylureas, meglitinides, GLP-1 analogs, GLP-1 antagonists and DPP4 inhibitors.
33. The method of clause 32, wherein the agent that increases the sensitivity of cells to insulin is a PPAR alpha, gamma, or delta agonist.
34. The method of any one of clauses 27-33, wherein the programmable therapy delivery module is configured to deliver an electrical signal having a frequency of at least 200 Hz.
35. The method of any one of clauses 27-33, wherein the programmable therapy delivery module is configured to deliver an electrical signal having a frequency of 500 Hz to 5 kHz, 1 kHz to 10 kHz or 10 kHz to about 80 kHz.
36. The method of any one of clauses 27-33, wherein the programmable therapy delivery module is configured to deliver a HFAC, low frequency stimulation or burst stimulation signal.
37. The method of clause 36, wherein current amplitudes of HFAC, low frequency stimulation or burst stimulation signal is between 0.1 mA to 20 mA.
38. The method of any one of clauses 36-37, wherein a pulse width for the low frequency stimulation signal or burst stimulation signal is between 0.01 ms to 10 ms.
39. The method of any one of clauses 36-37, wherein the frequency of the low frequency stimulation signal is from 0.01 Hz to 199 Hz, 0.1 to 10 Hz, 10 Hz to 30 Hz, 30 Hz to 100 Hz, 100 Hz to 199 Hz.
40. The method of any one of clauses 27-39, wherein the total volume of the implantable pulse generator will not exceed 0.5 in.³, 1 in.³, 1.5 in.³, 1.75 in.³, or 2.0 in.³.
41. The method of any one of clauses 27-40, wherein the combined total charge of the delivery of the HFAC and 1 Hz signal for 1-minute is less than 0.40 coulombs.
42. The method of any one of clauses 27-41, wherein the delivery of a 1 minute 5 kHz signal is at least 90% less than the same 5 kHz signal delivered for 4 hrs.
43. The method of any one of clauses 27-42, wherein the electrical signal is delivered between 1 second to 1 hour.
44. The method of any one of clauses 27-43, wherein the electrical signal is a burst signal and the burst signal is delivered between about 10 seconds to about 30 minutes.
45. The method of any one of clauses 27-44, wherein the system for inducing plasticity is delivered to a sub-diaphragmatic target.
46. The method of any one of clauses 27-45, wherein the system treats a diseases selected from a group comprising obesity, inflammation, autoimmune diseases, rejection of organ transplant, pancreatitis, hypertension, cancer and any combination thereof.
47. The method of any one of clauses 27-46, wherein the programmable therapy delivery module is configured to deliver an electrical signal having an off time of at least 30 minutes between a second electrical treatment application.
48. The method of any one of clauses 27-47, wherein the programmable storage and communication module is configured to store and communicate more than one therapy program, wherein each therapy program is different from one another, and is configured to be selected for communication.
49. The method of any one of clauses 27-48, wherein programmable therapy delivery module is configured to deliver a second therapy program comprising an electrical signal treatment applied to a second target, wherein the second target is a nerve or an organ.
50. The method of any one of clauses 27-49, wherein the second target nerve is the splanchnic nerve or the celiac branch of the vagus nerve, or the dorsal vagus nerve central to the branching point of the celiac nerve.
51. The method of any one of clauses 27-50, wherein the system delivers a signal in a closed loop system.

What is claimed is:

1. A system for altering plasticity in a target comprising:
at least two electrodes operably connected to an implantable pulse generator, wherein at least one of the electrodes is adapted to be placed on a target to alter the plasticity.

2. The system of claim 1, wherein the implantable pulse generator comprises a power module and a programmable therapy delivery module, wherein the programmable therapy delivery module is configured to deliver at least one therapy program comprising an electrical signal treatment applied to the target, wherein the electrical signal has a frequency selected to initiate activity on the target; and
an external component comprising a communication system and a programmable storage and communication module, wherein programmable storage and communication module is configured to store the at least one therapy program and to communicate the at least one therapy program to the implantable pulse generator and wherein the activity is a neural stimulation or a neural block.

3. The system of claim 2, wherein the electrical signal treatment is continuously applied to the target nerve.

4. The system of claim 2, wherein the electrical signal treatment is bursting neuromodulation applied to the target.

5. The system of claim 1, wherein the electrical signal has an on time and an off time, wherein the off time is selected to allow at least a partial recovery of the activity of the target nerve.

6. The system of claim 1, wherein the off time is configured to commence upon the detection of blood glucose levels between 80 mg/dL and 110 mg/dL.

7. The system of claim 1, where the communication system is selected from a group consisting of an antenna, blue tooth technology, radio frequency, WIFI, light, sound and combinations thereof.

8. The system of claim 1, wherein the at least one electrode is adapted to be placed on an organ selected from the spleen, stomach, duodenum, pancreas, liver and ileum.

9. The system of claim 1, wherein the at least one electrode is adapted to be placed at a target, wherein the target is a nerve selected from a group comprising a vagus nerve, a splanchnic nerve, a hepatic branch of a vagus nerve, a celiac branch of a vagus nerve and combinations thereof.

10. The system of claim 2, wherein the programmable therapy delivery module is configured to deliver an electrical signal having a frequency of at least 200 Hz.

11. The system of claim 2, wherein the programmable therapy delivery module is configured to deliver an electrical signal having a frequency of 500 Hz to 5 kHz, 1 kHz to 10 kHz or 10 kHz to about 80 kHz.

12. The system of claim 2, wherein the programmable therapy delivery module is configured to deliver a HFAC, low frequency stimulation or burst stimulation signal.

13.-27. (canceled)

28. A method of for altering plasticity in a target comprising: applying an electrical signal to a target nerve or organ of the subject having impaired glucose regulation using the system of claim 1.

29. (canceled)

30. The method of claim 28, further comprising administering an agent that improves glucose control.

31. The method of claim 30, wherein the agent increases the amount of insulin and/or increases the sensitivity of cells to insulin.

32. The method of claim 31, wherein the agent that increases the amount of insulin is selected from the group consisting of insulin, insulin analogs, sulfonylureas, meglitinides, GLP-1 analogs, GLP-1 antagonists, and DPP4 inhibitors.

33. The method of claim 32, wherein the agent that increases the sensitivity of cells to insulin is a PPAR alpha, gamma, or delta agonist.

34. The method of claim 28, wherein the programmable therapy delivery module is configured to deliver an electrical signal having a frequency of at least 200 Hz.

35. The method of claim 28, wherein the programmable therapy delivery module is configured to deliver an electrical signal having a frequency of 500 Hz to 5 kHz, 1 kHz to 10 kHz or 10 kHz to about 80 kHz.

36.-39. (canceled)

40. The method of claim 28, wherein the total volume of the implantable pulse generator will not exceed 0.5 in.³, 1 in.³, 1.5 in.³, 1.75 in.³, or 2.0 in.³.

41. The method of claim 28, wherein the combined total charge of the delivery of the HFAC and 1 Hz signal for 1-minute is less than 0.40 coulombs.

42.-50. (canceled)

51. The method of claim 28, wherein the system delivers a signal in a closed loop system.
